(19) United States
(12) Patent Application Publication
(10) Pub. No.: US 2016/0292319 A1
(43) Pub. Date: Oct. 6, 2016

(54) BODY SUPPORT CUSTOMIZATION BY GENERATION AND ANALYSIS OF A DIGITAL LIKENESS

(71) Applicant: Sealy Technology, LLC, Trinity, NC (US)

(72) Inventor: Darin T. Thomas, Salisbury, NC (US)

(73) Assignee: Sealy Technology, LLC, Trininty, NC (US)

(21) Appl. No.: 14/676,871

(22) Filed: Apr. 2, 2015

(51) Int. Cl. G06F 17/50; G06T 7/00; H04N 13/02; H04N 5/33; H04N 5/225

(52) U.S. Cl. CPC G06F 17/5009; H04N 5/33; H04N 5/2258; H04N 13/0257; H04N 13/0253; H04N 13/0271; G06T 7/0057; G06T 2200/04; G06T 2207/10024; G06T 2207/10028; G06T 2207/10048; G06T 2207/30196

(57) ABSTRACT

A system for generating a digital likeness of a user to model a customized body support includes a both scanning system including a body image scanner in communication with a processing system, a digital likeness generation module residing on the processing system and configured to operate the body scanning system to generate the digital likeness of the user in a field of view of the body image scanner, and a both support simulation module in communication with the digital likeness generation module and configured to receive the digital likeness of the user and digitally model a body support customized to the digital likeness of the user. A method of digitally generating a customized body support is also provided.

### FIELD OF THE INVENTION

The present invention relates to machines and processes for the customization of a body support. More specifically, the present invention relates to generation of a digital likeness, analysis of the likeness, and based on the analysis, creating a customized body support.

### BACKGROUND

Body supports are generally known in the art, and often include one or more deformable elements provided to support one or more body parts of a human or animal. A body support may include, but is not limited to, a mattress, pillow, or cushion, including those for use in beds, seats, or chairs. A body support may be desired shape or size suitable to support a portion, up to and including the entirety, of the user.

Known body supports may be constructed of a single layer of material. For example, a both support ma be constructed of a single layer of natural material, such as cotton, down, and other natural materials, foam and other synthetic materials, devices and objects such as air bladders, metal or plastic springs, and the like. Still other body supports may be constructed of multiple layers of any of these materials. For example, multi-layer body supports can be made of two or more layers of synthetic foams, such as polyurethane viscoelastic or non-viscoelastic foam, latex foam, and/or other foam materials.

Known single and multi-layer body supports are generally mass produced. During mass production, each layer of the body support is typically constructed of a homogeneous material based on a uniform pattern or design. Accordingly, each layer is typically constructed to have the same dimensions and physical properties across the body support.

Known body supports are also riot generally customized to the body type of a user. Commercially available body supports are typically sold by a level of firmness, for example firm, plush, euro plush, and pillow top. Some other known body supports provide firmness adjustment, for example by providing internal air chambers that may be inflated or deflated with air to respectfully increase or decrease the firmness. While these known body supports allow a user to select or adjust firmness, these body supports are generally not manufactured so that they are customized to the user. Instead, such adjustable body supports typically require the user to adjust the body support to the user's preferences. This often limits the degree to which the body support can be adjusted to the user. Also, such body supports often have limited firmness customization across the body support, and are often not customized to the body type of the user.

### SUMMARY OF THE INVENTION

The invention provides, in one aspect, a system for generating a digital likeness of a user to model a customized body support. The system includes a body scanning system including a body image scanner in communication with a processing system, a digital likeness generation module residing on the processing system and configured to operate the body scanning system to generate the digital likeness of the user in a field of view of the body image scanner, and a body support simulation module in communication with the digital likeness generation module. The body support simulation module is configured to receive the digital likeness of the user and digitally model a body support customized to the digital likeness of the user.

The invention provides, in another aspect, a method of digitally generating a customized body support including generating a digital likeness of a user and generating a digital model of a body support customized to the user based on the digital likeness.

Other features and aspects of the invention will become apparent by consideration of the following detailed description and accompanying drawings.

### BRIEF DESCRIPTION OF THE DRAWINGS

FIG. 1 is a schematic diagram of a body scanning system for generating a digital likeness of a user in accordance with an embodiment of the invention.

FIG. 2 is a schematic diagram of a processing system used with the body scanning system of FIG. 1.

FIG. 3 is a flow diagram of a digital likeness generation application for use with the body scanning system of FIG. 1.

FIG. 4 is a flow diagram of a body support simulation application that uses the digital likeness of the user captured by the body scanning system of FIG. 1 to digitally model a customized body support optimized for relative comfort and support, improving sleep quality.

FIG. 5 is an isometric view of a portion of an example of a customized body support resulting from the body support simulation application of FIG. 4.

Before any embodiments of the present invention are explained in detail, it should be understood that the invention is not limited in its application to the details or construction and the arrangement of components as set forth in the following description or as illustrated in the drawings. The invention is capable of other embodiments and of being practiced or of being carried out in various ways. It should be understood that the description of specific embodiments is not intended to limit the disclosure from covering all modifications, equivalents and alternatives falling within the spirit and scope of the disclosure. Also, it is to be understood that the phraseology and terminology used herein is for the purpose of description and should not be regarded as limiting.

### DETAILED DESCRIPTION

Embodiments of the present invention illustrated in the Figures and disclosed herein are generally directed to a system comprising a body scanning system 100, a digital likeness generation application 300, and a both support simulation application 400. The system 100 and applications 300, 400 provide as use 200 the opportunity to have a customized body support 500 manufactured based at least in part upon the generation of a digital likeness of the user's body, and an analysis and simulation of the digital likeness to model the customized body support 500. The customized body support 500 can be matched to the user's body type and sleep preferences to improve a relative comfort and sleep quality of the user 200.

For ease of discussion and understanding, the following detailed description refers to a body support. The features described herein in association with the body support are applicable to any suitable element provided to support one or more body parts of a human or animal. Accordingly, the term “body support” may include but is not limited to a mattress, mattress topper, overlay, futon sleeper sofa, cushion, seat cushion, seat back, pillow, neck pillow, leg spacer pillow, eye mask, or any other element provided to support as portion up to and including the entirety, of as human or animal. In addition, the body support may be any suitable or desired size or shape.

It should be appreciated that the term “relative comfort” of a user is directed to include how comfortable or physically content a user is in association with the body support at any given moment in time. The term relative comfort may generally be as subjective level of physical comfort felt by a user at a given moment while using the body support, or after the conclusion of use of the buds support. Relative comfort may also change from moment to moment, and may be unique to one or more different users.

In addition, it should be appreciated that the terms “physical properties” of the body support is inclusive of properties of the body support that may be modified, controlled, or adjusted during manufacturing. Physical properties of the body support include, but are not limited to, firmness, density, sag-factor, air flow, resilience, hardness of material, and compression.

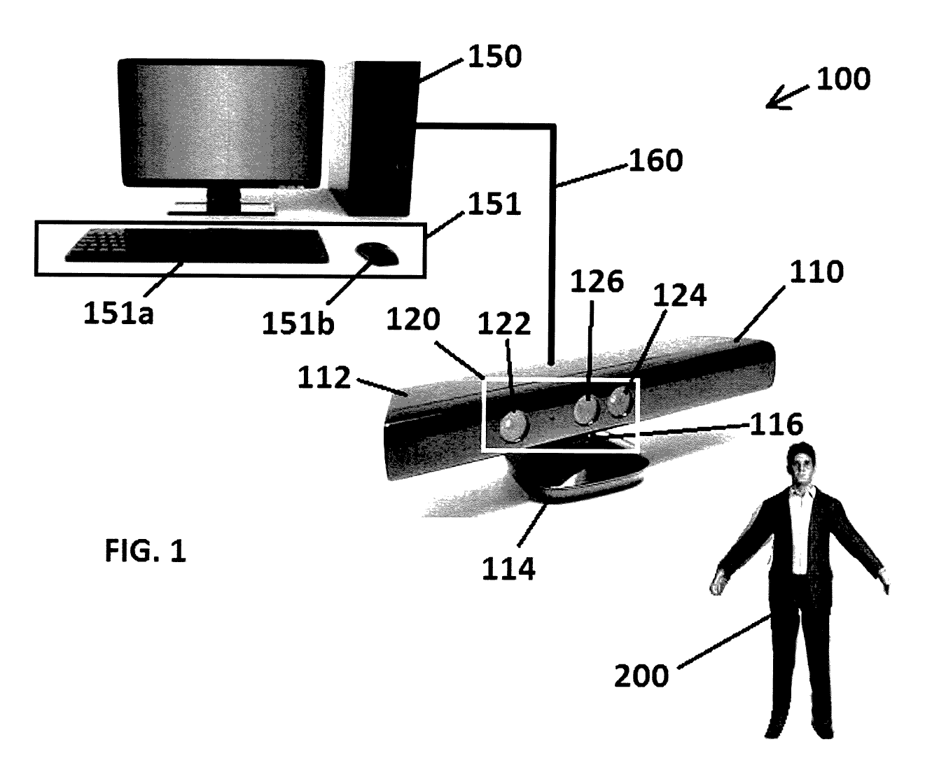

Referring now to the figures, FIG. 1 illustrates an embodiment of a body scanning system 100. The body scanning system 100 includes a body image scanner 110 in communication with a processing system 150 through a communication link 160. The body image scanner 110 scans and captures one or more digital images of a body of a person or user 200, and works in conjunction with the processing system 150 to generate a digital likeness of the body of the user 200.

The body image scanner 110 includes a housing 112 connected to a base 114 by a motorized tilt assembly 116. The tilt assembly 116 enables the housing 112 to rotate about the base 114 in at least a side-to-side direction (or along an x-axis) and to pivot about the base 114 in at least an up-and-down direction (or along a y-axis). In other embodiments, the body image scanner 110 may optionally include the base 114 and/or motorized tilt assembly 116.

The housing 112 contains a plurality of cameras 120 and associated operational electronics for scanning and capturing one or more digital images of the body of the user 200. The cameras 120 include a three-dimensional (3D) depth sensor defined by an infrared (IR) emitter or projector 122 and an infrared (IR) depth sensor 124. The IR emitter 122 and IR depth sensor 124 work in conjunction to construct a depth image or depth map. The IR emitter 122 projects infrared (IR) light beams outward from the body image scanner 110. The IR depth sensor 124 detects reflection and deformation of the IR light beams to ascertain different distances between the object and the sensor 124. The distance data is used to construct the depth image, providing a point cloud or three-dimensional representation of the object and environment in a field of view of the IR emitter 122 and sensor 124. The IR emitter 122 and depth sensor 124 may be electrically connected to a complimentary metal-oxide semiconductor (CMOS) sensor (not shown) to allow for depth image construction of the object and environment regardless of lighting conditions.

The cameras 120 also include a color sensor or red, green, blue (RGB) camera 126. The RGB camera 126 captures a three color image of the object and environment in a field of view of the camera 126. It should be appreciated that the IR emitter 122 and depth sensor 124, and the RGB camera 126, can capture digital still images, such as a digital photographs, and digital video.

In FIG. 1, the body image scanner 110 is illustrated as a KINECT motion sensing input device available from MICROSOFT CORPORATION (headquartered in Redmond, Wash.). The KINECT is provided for purposes of illustration only. In other embodiments, the body scanning system 100 may use other suitable known or future developed scanning devices for scanning the body of the user 200.

The body image scanner 110 is electrically connected to the processing system 150 through the communication link 160. The communication link 160 provides a pathway for communication between the body image scanner 110 and the processing system 150. The communication link 160 is illustrated as a Category 5 or Cat5 cable. However, in other embodiments, the communication link 160 may be any suitable communications protocol or pathway, including, but not limited to, wireless communication, communication via transmission control protocol/internet protocol (TCP/IP), Ethernet, or universal serial bus (USB), and the like.

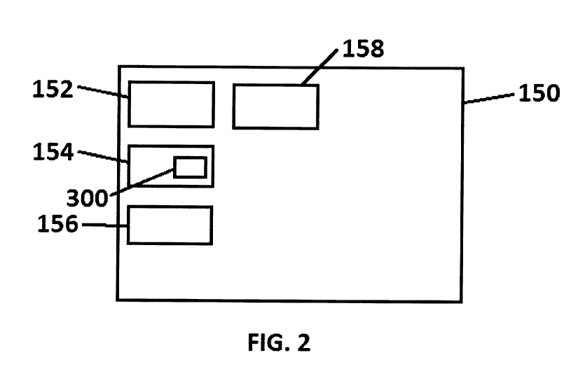

The processing system 150 is a programmable computer system in communication with the body image scanner 110. The processing system 150 includes a user input portion 151, illustrated in FIG. 1 as a keyboard 151a and a mouse 151b. Referring now to FIG. 2, the processing system 150 includes random access memory (RAM) 152, a computer readable storage medium or hard drive 154, a processor 156, and a port or Ethernet port 158 for connecting the processing system 150 to a distributed network, such as the Internet. The processing system 150 may include executable instructions (or a computer readable code) to receive and store images and/or video of the user 200 acquired by the body image scanner 110, to construct a digital likeness of the user 200, and to store the digital likeness. In one or more examples of embodiments, the processing system 150 may be any known or future developed programmable computer processor system suitable fir communication with the body image scanner 120, and to process and analyze any and all data as disclosed herein. For example, the processing system 150 may be an XBOX (available from MICROSOFT CORPORATION) or a PLAYSTATION (available from SONY CORPORATION, headquartered in Tokyo, Japan) brand entertainment platform. In these embodiments, the user input portion 151 may be a controller or other device to interact with the processing system 150. Additionally, and depending on the brand of the processing system 150, the body image scanner 110 may be, for example an XBOX KINECT system or another system with similar functionality. Further, in one or more examples, the computer readable storage medium 154 may include any data storage device which can store data that can be thereafter read by a computer system. Examples of computer readable storage medium 154 may include read-only memory, CD-ROM, CD-R, CD-RW, DVD, DVD-RW, magnetic tapes, Universal Serial Bus (USB) flash drive, or any other magnetic, optical or other suitable data storage device. The computer readable storage medium 154 may also be distributed over a network coupled to or in communication with the processing system 150 so that any executable instructions are stored and/or executed in a distributed fashion.

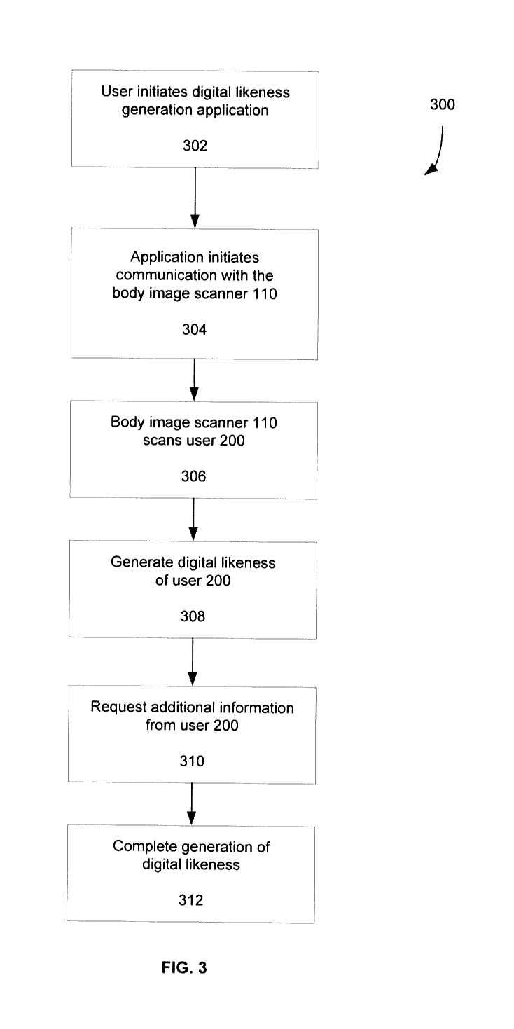

FIG. 3 illustrates an example of a digital likeness generation application 300 using the body scanning system 100 described herein. The application or module 300 includes a series of processing instructions or steps that are depicted in flow diagram form. The application 300 may be distributed and stored on the computer readable storage medium 154 of the processing system 150 (as shown in FIG. 2), and/or can be accessible for execution from a remote location, such as through a web portal, web site, or generally over the Internet.

Referring to FIG. 3, the process begins at step 302, where the body scanning system 100 is in a powered on state and the user initiates the digital likeness generation application 300. The application 300 is initiated, for example, by the user activating an executable file. Next, at step 304, the application initiates communication with the body image scanner 110.

At step 306, the body image scanner 110 scans the user 200. The scan at step 306 may include the user 200 performing one or more motions to determine a relative flexibility and/or a range of motion of the user 200. In addition, the user 200 may be prompted to turn in relation to the body image scanner 110 at predetermined intervals to capture images of the body of the user 200 from different views. For example, the user 200 may face towards the body image scanner 110, face away from the body image scanner 110, and/or present one shoulder closer to the body image scanner 110 than the other shoulder (in order to capture images of one or both sides of the user's body). In other embodiments, the scanning step may include a thermal scan of the user to generate a user's thermal signature. This information may be used to correlate factors such as a temperature around the user's face, the user's hands, feet, or other extremities, or other body locations of the user.

Next, at step 308, the application 300 generates a digital likeness of the user 200. The digital likeness is a three-dimensional digital representation of the user 200 defined by the point cloud of the user 200 captured by the body image scanner 110.

At step 310, the application 300 may request a User 200 to provide additional information to complete the digital likeness. The additional information may include size or dimension information of the user 200, including chest size, waist size, inseam, arm length, neck circumference, or head circumference. The additional information may also or alternatively include sleep preferences, such as one or more preferred sleeping positions (e.g. fetus, log, yearner, soldier, free-fall, starfish, or other) health problems (e.g. restless leg syndrome, pinched nerves, or overall body fatigue), preferred sleeping temperature, whether there is a sleeping partner, and the sleeping partner's preferences.

Next, at step 312, generation of the digital likeness is complete. The digital likeness and any additional information may be stored locally at the processing system 150, or uploaded over a network (or the Internet) to a remote computer readable storage medium for analysis and predictive simulation.

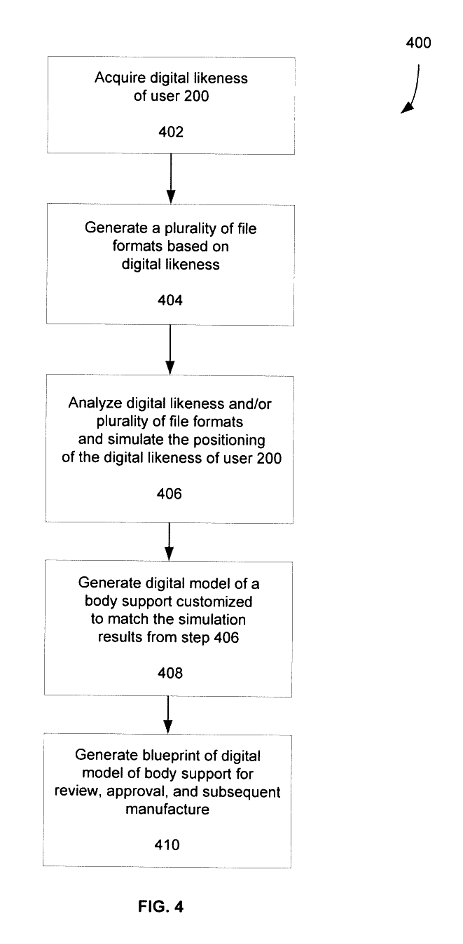

FIG. 4 illustrates an example of a body support simulation application 400 using the digital likeness generated by the body scanning system 100. The application or module 400 also includes a series of processing instructions or steps that are depicted in flow diagram form. The application 400 may be distributed and stored on the computer readable storage medium 154 of the processing system 150, and/or can be executed at a location remote from the body scanning system 100.

Referring to FIG. 4, at step 402 the application 400 acquires the digital likeness of the user. The acquisition may be by direct communication with application 300, or may be through a user initiated upload or transfer of the digital likeness.

Next, at step 404, the digital likeness is used to generate a plurality of file formats. The file formats may include using the digital likeness to generate a stick figure of the user 200, a body shape overlaying the stick figure, and/or a musculoskeletal representation of the user 200.

At step 406, the digital likeness, the plurality of file formats, and/or the additional information provided by the user 200, including preferred sleep positions, are analyzed and used to virtually simulate and predict a relative comfort of the user 200. The simulation can consider minimization of pressure points along the digital likeness of the user 200, characterize, muscle forces present, and/or improve spinal alignment to improve the relative comfort of the user 200. The simulation results in a positioning of the digital likeness of the user 200 that optimizes relative comfort for sleep.

At step 408, a simulated body support or digital model is constructed to provide support that matches or conforms to the simulation results at step 406. More specifically, the simulated body support provides physical support for the user 200 for optimal sleep, while also improving relative comfort of the user 200. The combination provides a body support tailored to the user 200, and optimized to provide support and relative comfort to improve sleep quality. The simulated body support is customizable along the length, width, and thickness of the body support (e.g. x-direction, y-direction, and z-direction) to provide a level of support sufficient to provide the resulting relative comfort of the user 200 from the simulation at step 406. The simulated body support may include variations on springs, foams, fibers, sensors, and any other materials used to construct the body support. In addition, the body support may include multiple and different layers, densities, and/or compositions of foam (or other materials) along the length, width, and thickness of the body support.

Next, at step 410, the process generates a blueprint of the simulated body support. The blueprint provides design, materials, and/or manufacturing details of the customized body support for subsequent manufacture. The blueprint is provided to the user 200 or manufacturer for review, approval, and later use. For example, the user may immediately review and approve manufacturing of a body support based on the blueprint. Otherwise, the system 400 may maintain the blueprint for later review and manufacturing approval by the user 200.

It should be appreciated that the body scanning system 100 and digital likeness generation application 300 may be accessible by the user 200 at to store front or point of sale. Alternatively, the system 100 and application 300 may be accessible for use in the privacy of the user's 200 home.

Similarly, the body support simulation application 400 may operate at a remote location, such as a website or server hosted by the body support manufacturer, or may be distributed to the body scanning system 100 for operation. In embodiments where the application 400 operates on the body scanning system 100, the results from the blueprint generation at step 410 may be communicated to the manufacturer for manufacture.

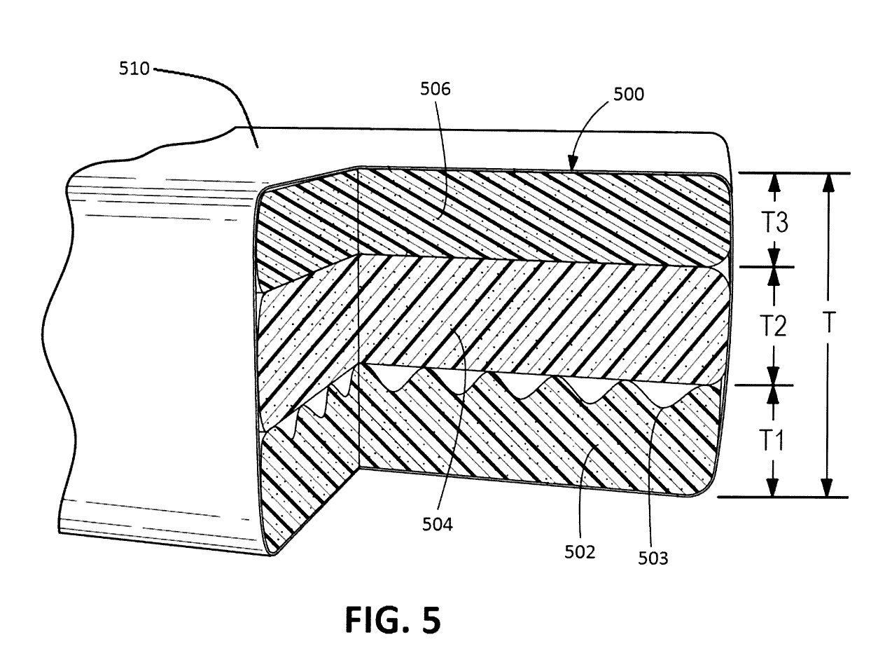

FIG. 5 illustrates an example of a portion of a customized body support 500 manufactured after the simulation from application 400. The body support 500 includes a plurality of layers 502, 504, 506. A first or base liner 502 defines a first thickness T1. A second layer 504 that defines a second thickness T2 adjoins or is stacked on the first layer 502. A third layer 506 that defines a third thickness T3 adjoins or is stacked on the second layer 504. The layers 502, 504, 506 cooperatively define a thickness T of the body support 500. Each of the illustrated layers 502, 504, 506 is formed of a homogeneous material within each layer, but each layer 502, 504, 506 has a different foam formulation than the other layers 502, 504, 506. However, in alternative embodiments the one or more layers 502, 504, 506 may have similar formulations, and/or one or more layers 502, 504, 506 may have different formulations within each layer. One or more layers 502, 504. 506 may also include additives, such as phase change material. Further, in some embodiments the one or more layers 502, 504, 506 may include materials other than foam, such as fibers, springs, fabrics, or other materials.

The first layer 502 of the illustrated, embodiment of FIG. 5 includes a pattern 503, illustrated as a plurality of projections and convolutions. In alternative embodiments of the body support 500, the support 500 may include fewer than three or more than three layers. In addition, in some embodiments none of the layers, or a plurality of the layers of the body support 500 may include a pattern. Further, the pattern may be provided in any of the layers in order to attain the desired physical properties or relative comfort. In other embodiments, each layer 502, 504, 506 may include different foam formulations within each layer.

An outer barrier 510 encases the body support 500. The outer barrier 510 may be a fire barrier, ticking cover, or other material that may encase a portion (or the entirety) of the internal foam layers, resulting in the finalized customized body support 500 product.

The body scanning system 100, digital likeness generation application 300, and body support simulation application 400 provide for body support customization based on the body type of the user 200, in addition to other factors. By generating a digital likeness of the user 200 and then simulating an appropriately matched body support, customization is optimized to increase relative comfort of the user 200 while also tailoring physical support to the user 200. In the end, the user 200 receives a body support matched to the user's body type and/or sleep preferences that is optimized to improve relative comfort and sleep quality. These and other advantages may be realized from one or more embodiments of the system, processes, and associated body supports disclosed herein.

What is claimed is:

1. A system for generating a digital likeness of a user to model a customized body support, the system comprising:
a body scanning system including a body image scanner in communication with a processing system;
a digital likeness generation module residing on the processing system and configured to operate the body scanning system to generate the digital likeness of the user in a field of view of the both image scanner; and
a body support simulation module in communication with the digital likeness generation module, the body support simulation module configured to receive the digital likeness of the user and digitally model a body support customized to the digital likeness of the user.

2. The system of claim 1, wherein the body support simulation module generates a blueprint of the customized body support for manufacturing.

3. The system of claim 1, wherein the body image scanner includes a three dimensional depth sensor.

4. The system of claim 3, wherein the three dimensional depth sensor includes an infrared emitter and an infrared depth sensor.

5. The system of claim 5, wherein the infrared emitter and the infrared depth sensor define the field of view of the body image scanner, and are configured to generate a point cloud of the user provided in the field of view of the body image scanner.

6. The system of claim 3, wherein the body image scanner includes a color sensor configured to capture color images in the field of view of the body image scanner.

7. The system of claim 6, wherein the color sensor includes a red, green, blue camera.

8. The system of claim 1, wherein the digital likeness generation module resides on a computer readable storage medium associated with the processing system.

9. The system of claim 1, wherein the processing system is a MICROSOFT XBOX, and the body image scanner is an XBOX KINECT.

10. The system of claim 8, wherein the body support simulation module resides on a second computer readable storage medium remote from the body scanning system.

11. The system of claim 1, wherein the digital likeness generation module communicates with the body support simulation module through the Internet.

12. The system of claim 1, wherein the body support simulation module is configured to digitally model a body support customized to improve a relative comfort and physical support based on the digital likeness of the user and a sleep preference of the user.

13. The system of claim 13, wherein the sleep preference is selected from the group consisting of a preferred sleeping position, a user health problem, a preferred sleeping temperature, the existence of a sleeping partner, and the sleeping partner's sleep preference.

14. A method of digitally generating a customized body support, the method comprising:
generating a digital likeness of a user; and
generating a digital model of a body support customized to the user based on the digital likeness.

15. The method of claim 15, further comprising generating a blueprint of the digital model of the body support for manufacturing.

16. The method of claim 15, further comprising generating a plurality of file formats from the digital likeness of the user, after the step of generating the digital likeness of the user.

17. The method of claim 17, wherein the file formats include a stick figure of the user, a body shape overlaying the stick figure of the user, or a musculoskeletal representation of the user.

18. The method of claim 15, wherein generating the digital likeness of the user includes scanning the user with a body image scanner to generate a point cloud of the user.

19. The method of claim 15, further comprising requesting a sleep preference from the user, after the step of generating the digital likeness of the user.

20. The method of claim 20, wherein the sleep preference is selected from the group consisting of a preferred sleeping position, a user health problem, a preferred sleeping temperature, the existence of a sleeping partner, and the sleeping partner's sleep preference.

21. The method of claim 20, further comprising:
analyzing the digital likeness and at least one sleep preference; and
generating the digital model of the body support customized to the user based on the analysis of the digital likeness and the at least one sleep preference.

22. The method of claim 15, wherein generating the digital likeness of the user includes scanning the user with a body image scanner while the user faces towards the body image scanner, and scanning the user with the body image scanner while the user faces away from the body image scanner.

23. The method of claim 23, wherein generating the digital likeness of the user includes scanning the user with the body image scanner while the user has one shoulder closer to the body image scanner than the other shoulder.
